(19) United States
(12) Patent Application Publication
(10) Pub. No.: US 2018/0322507 A1
(43) Pub. Date: Nov. 8, 2018

(54) WARRANTY MANAGEMENT SYSTEM AND METHODS OF PERFORMING THE SAME

(71) Applicant: Champion Medical Technologies, Inc., Lake Zurich, IL (US)

(72) Inventors: Peter I. Casady, Chicago, IL (US); Tom Casady, Phoenix, AZ (US); Charity Carney, Phoenix, AZ (US)

(21) Appl. No.: 15/971,847

(22) Filed: May 4, 2018

Related U.S. Application Data
(60) Provisional application No. 62/502,265, filed on May 5, 2017.

(51) Int. Cl. G06Q 30/00; G06Q 10/10; G16H 40/40; G06Q 30/02

(52) U.S. Cl. CPC G06Q 30/012; G06Q 30/0283; G16H 40/40; G06Q 10/10

(57) ABSTRACT

The present disclosure relates to a warranty management system. In an embodiment, the warranty management system includes an information gathering unit that gathers information on a medical device, a warranty tracking unit that gathers warranty information on the medical device, submits a warranty claim and tracks the status of the warranty claim, a credit calculation unit that determines the amount of a credit for the warranty claim, and a payment distribution unit that generates a list of payees based on the warranty information and generates a payment to each payee.

### CROSS-REFERENCE TO RELATED APPLICATIONS

This application claims the benefit of and priority from U.S. Provisional Patent Application Ser. No. 62/502,265, filed May 5, 2017, which is hereby fully incorporated herein by reference.

### FIELD OF THE INVENTION

The present invention generally relates to a system and method for managing recalls of medical devices.

### BACKGROUND OF THE INVENTION

When a medical device is extracted from a patient due to a failure, the patient, the hospital and/or the insurance company may be entitled to a credit, however, the device must be submitted to the manufacturer as a warranty claim to receive the credit. Further, the credit typically will be divided amongst the hospital, patient and insurance company.

Current known processes are unacceptable to health care providers because there is no system that tracks the submission of credits, receipt of credits and division of credits to different parties. Known methods and systems for managing warranty credits fail to perform these steps.

Accordingly, there is a need for an improved system for managing recalls of medical devices.

### SUMMARY OF THE INVENTION

A warranty management system including an information-gathering unit that gathers information on a medical device; a warranty tracking unit that gathers warranty information on the medical device and submits a warranty claim and tracks the status of the warranty claim; a credit calculation unit that determines the amount of a credit for the warranty claim; and a payment determination unit that generates a list of payees based on the warranty information and generates a payment to each payee.

### BRIEF DESCRIPTION OF THE DRAWINGS

Details of the present invention, including non-limiting benefits and advantages, will become more readily apparent to those of ordinary skill in the relevant art after reviewing the following detailed description and accompanying drawings, wherein:

FIG. 1 depicts a block diagram of a warranty tracking system suitable for use with the methods and systems consistent with embodiments of the present invention;

FIG. 2 shows a more detailed depiction of the warranty tracking unit of FIG. 1;

FIG. 3 shows a more detailed depiction of the computers of FIG. 1;

FIG. 4 depicts a schematic representation of the process carried out by the system of FIG. 1;

FIG. 5 depicts a process flow diagram of a system in accordance with an embodiment of the present invention;

FIG. 6 depicts exemplary graphical user interface of a credit calculation display;

FIG. 7 depicts an exemplary graphical user interface of a resolve claim screen; and

FIG. 8 depicts an exemplary graphical user interface of an analytics screen.

### DETAILED DESCRIPTION

For the purposes of promoting and understanding the principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed and illustrated herein are contemplated as would normally occur to one of skill in the art to which this disclosure relates.

The present invention discloses a system that gathers information concerning the recall of a medical device formerly implanted in a patient, generates a claim on the device and sends the device to the manufacturer under a warranty claim. The system tracks the recalled device as it moves through the warranty process and allocates payment of any credit to products identified by predetermined payment rules.

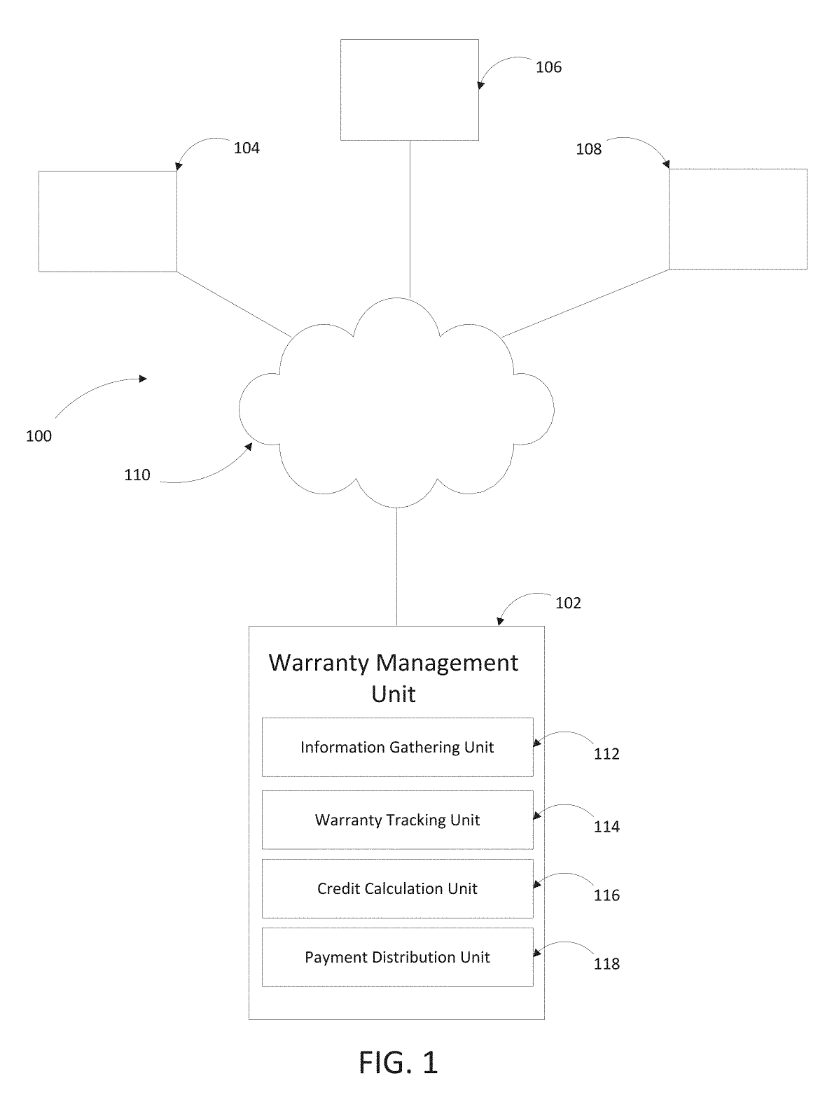

FIG. 1 depicts a block diagram of a warranty management system 100 suitable for use with the methods and systems consistent with the present invention. The warranty management system unit 100 comprises a plurality of computers 102, 104, 106 and 108 connected via a network 110. The network 110 is of a type that is suitable for connecting the computers for communication, such as a circuit-switched network or a packet-switched network. Also, the network 110 may include a number of different networks, such as a local area network, a wide area network such as the Internet, telephone networks including telephone networks with dedicated communication links, connection-less networks, and wireless networks. In the illustrative example shown in FIG. 1, the network 110 is the Internet. Each of the computers 102, 104, 106 and 108 shown in FIG. 1 is connected to the network 110 via a suitable communication link, such as a dedicated communication line or a wireless communication link.

In an illustrative example, computer 102 contains specialized instructions within memory that, when executed using the central processing unit (CPU) of the computer 102, implements a warranty management system 100 that includes an information gathering unit 112, a warranty tracking unit 114, a credit calculation unit 116 and a payment distribution unit 118. The number of computers and the network configuration shown in FIG. 1 are merely an illustrative example. Further, as will be clear to one of skill in the art, each element of the warranty management system 100 can alternatively be implemented using specialized computing hardware appropriately configured to perform the steps described below. Similarly, one having skill in the art will appreciate that the warranty management system 100 may include a different number of computers and networks. For example, computer 102 may include the information gathering unit 112 as well as one or more of the warranty tracking unit 114 and payment distribution unit 118. Further, the credit calculation unit 116 may reside on a different computer than computer 102.

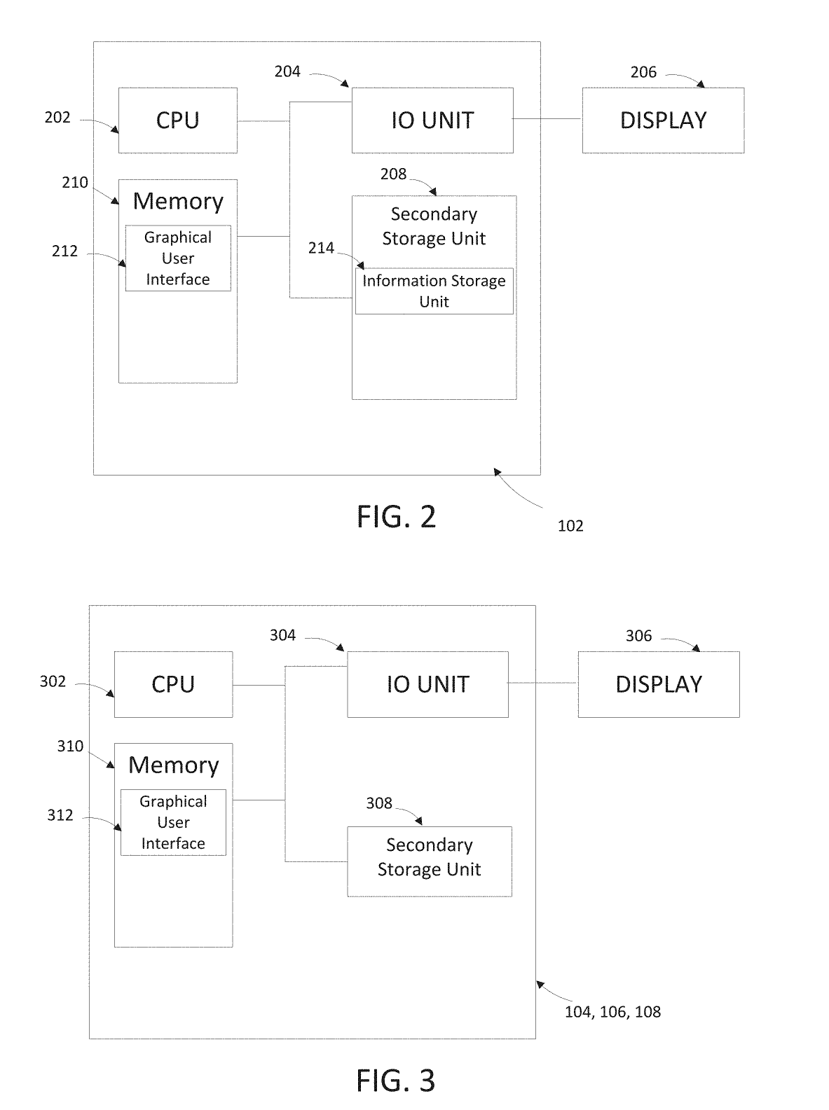

FIG. 2 shows a more detailed depiction of the computer 102. Computer 102 comprises a CPU 202, an input/output (IO) unit 204, a display device 206 communicatively coupled to the IO unit 204, a secondary storage unit 208, and a memory 210. The CPU 202 may further comprise standard input devices such as a keyboard, a mouse, a digitizer, or a speech processing means (each not illustrated).

The computer 102's memory 210 includes a graphical user interface (GUI) 212 which is used to gather information from a user via the display device 206 and I/O unit 204 as described herein. The GUI 212 includes any user interface capable of being displayed on a display device 206 including, but not limited to, a web page, a display panel in an executable program, or any other interface capable of being displayed on a computer screen. The GUI 212 may also be stored in the secondary storage unit 208. In one embodiment consistent with the present invention, the GUI 212 is displayed using commercially available hypertext markup language (HTML) viewing software such as, but not limited to, Microsoft Internet Explorer, Google Chrome or any other commercially available HTML viewing software. The secondary storage unit 208 may include an information storage unit 214. The information storage unit 214 may be a rational database such as, but not including Microsoft's SQL, Oracle or any other database.

FIG. 3 shows a more detailed depiction of the computers 104, 106 and 108. Each computer 104, 106 and 108 comprises a central processing unit (CPU) 302, an input/output (I/O) unit 304, a display device 306 communicatively coupled to the I/O unit 304, a secondary storage unit 308, and a memory 310. Each computer 104, 106 and 108 may further comprise standard input devices such as a keyboard, a mouse, a digitizer, or a speech processing means (each not illustrated).

Each computer 104, 106 and 108's memory 310 includes a graphical user interface (GUI) 312 which is used to gather information from a user via the display device 306 and I/O unit 304 as described herein. The GUI 312 includes any user interface capable of being displayed on a display device 306 including, but not limited to, a web page, a display panel in an executable program, or any other interface capable of being displayed on a computer screen. The GUI 312 may also be stored in the secondary storage unit 308. In one embodiment consistent with the present invention, the GUI 312 is displayed using commercially available hypertext markup language (HTML) viewing software such as, but not limited to, Microsoft Internet Explorer, Google Chrome or any other commercially available HTML viewing software.

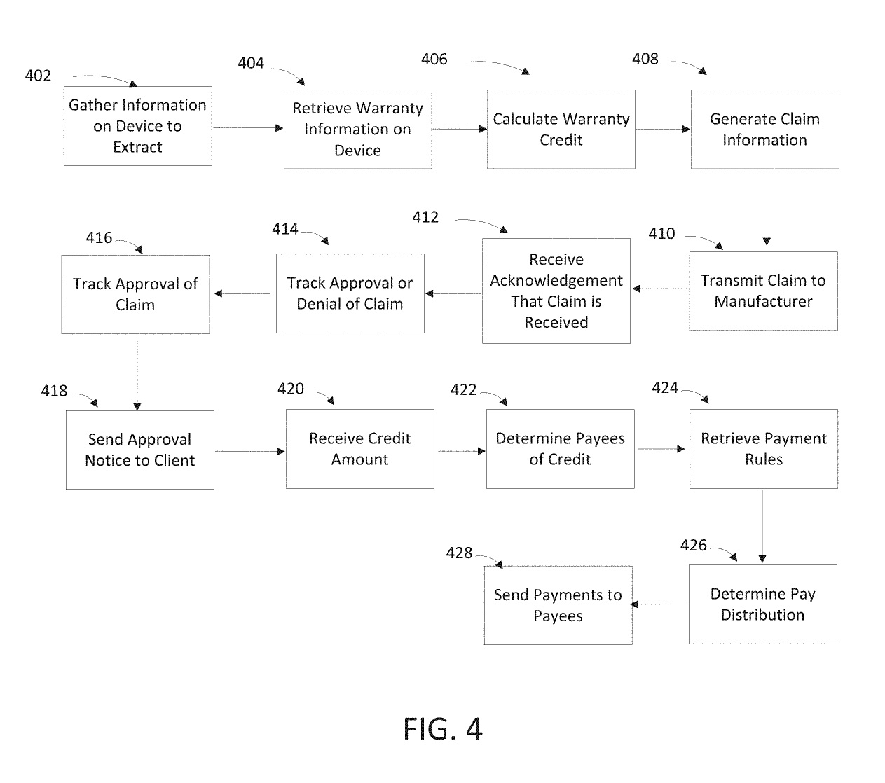

FIG. 4 depicts a schematic representation of a process performed by the warranty management system 100 in accordance with an embodiment of the present invention. The process begins with step 402, where the information gathering unit 112 gathers information on a medical device implanted in a patient. The information may comprise one or more of the manufacturer name, model number, and serial number for the medical device. The information can be gathered using physical methods such as wirelessly reading information from the medical device via a wireless communication device. In one embodiment, the information gathering unit 112 transmits a request for information concerning the medical device from an external data source based on identification information of the patient. The patient information may be provided by a user (for example, a medical care provider treating the patient) and may include, for example, personally identifiable information for the patient as well as an identification of a specific medical device implanted in the patient.

After gathering information on a specific medical device, the information gathering unit 112 retrieves warranty information on the medical device in step 404. In one embodiment, the information gathering unit 112 may retrieve the warranty information from a database in the secondary information storage unit 214 of the warranty management system 100. In another embodiment, the information management system 100 may retrieve the warranty information from an external source, such as the manufacturer of the device. The external source may be located remotely from the warranty management system 100 and may be accessed, for example, using the Internet.

In step 406, the warranty tracking unit 114 determines if a warranty applies to the medical device based on the received warranty information. This determination may be made, for example, by comparing the present date to the date on which the medical device was manufactured, sold, or implanted in the patient. In an embodiment, the user is alerted via GUI on the display device 206 if the warranty tracking unit 114 determines that the warranty appears to have expired and provides the option of either proceeding or terminating the claim submission process. In an alternative embodiment, if a warranty does not apply, the process automatically ends without user interaction. If a warranty does apply, the credit calculation unit 116 determines an estimated credit that may be received based on payment information related to the medical device and warranty information before proceeding with the process.

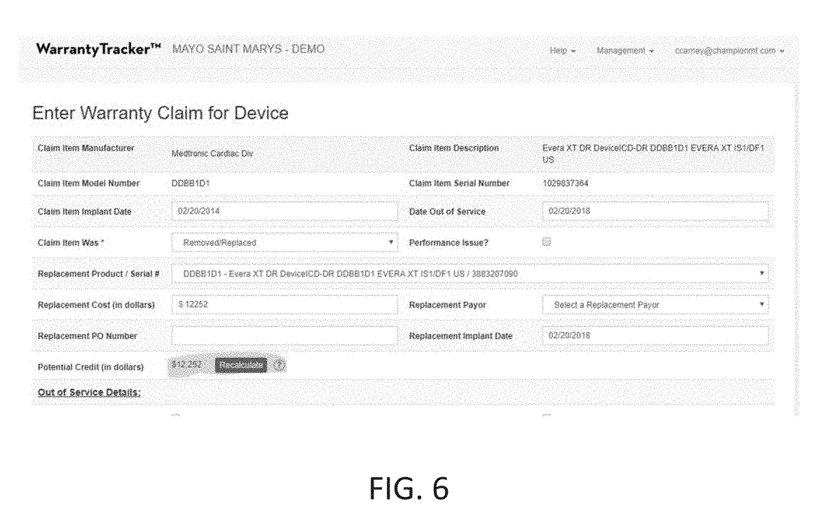

In an embodiment, the display device 206 may display the estimated credit, along with other information related to the claim, to a user using the GUI on the display device 206 for review and approval. An exemplary depiction of the GUI displayed on the display device 206 is provided in FIG. 6. As shown, in an embodiment the GUI provides information regarding the specific medical device that is the subject of the claim (e.g., manufacturer name, model number, description, and serial number), information regarding the date on which the medical device was implanted, the status of the medical device (i.e., whether it was removed or replaced), and the date on which the medical device was removed from service, information regarding the replacement product (if any), and the cost of the replacement. In addition, the GUI displays the estimated credit calculated in step 406. Optionally, the user may request that the estimated credit be recalculated (for example, after editing the claim information if any of the displayed information is incorrect). In an alternative embodiment, the method proceeds without requiring user interaction.

In step 408, the warranty tracking unit 114 generates a claim for a warranty payment based on warranty information retrieved from the information gathering unit 112 and the estimated credit. In an embodiment, the display device 206 may display the claim information to a user using the GUI on the display device 206 for review and approval prior to submission of the claim.

In step 410, the warranty claim is transmitted to the medical device manufacturer for processing. The warranty tracking unit 114 may also generate shipping information related to the location to send the medical device based on the warranty information. In one embodiment, the warranty tracking unit 114 interfaces with the shipping provider to confirm shipment and receipt of the medical device with the manufacturer.

In step 412, the warranty tracking unit 114 receives a confirmation of the receipt of the claim and the medical device from the medical device manufacturer. In the event a confirmation is not received within a predetermined period of time, the warranty tracking unit 114 may transmit a request to the manufacturer requesting a status of the warranty claim. Upon receipt of the warranty claim, the manufacturer may transmit an identifier related to the warranty claim to the warranty tracking unit 114. Upon receipt of the identifier, the warranty tracking unit 114 will store the identifier in the secondary storage unit 208 of the warranty management system 100.

In step 414, the warranty tracking unit 114 tracks the approval or denial of the warranty claim. In one embodiment, the warranty tracking unit 114 transmits requests to the manufacturer at regular intervals requesting status information on the warranty claim. In step 416, the warranty tracking unit 114 receives an approval of the warranty claim. In step 418, the warranty tracking unit 114 transmits a notification of approval to the client. In one embodiment, the warranty tracking unit 114 may notify the client using an e-mail message. Upon approval, the warranty tracking unit 114 registers the warranty claim as being approved for the specific medical device associated with the warranty. If the claim is denied, the warranty tracking unit 114 notifies the client of the denial and closes the claim.

In step 420, the warranty tracking unit 114 receives the amount of credit provided under the warranty from the manufacturer. In one embodiment, the warranty tracking unit 114 will compare the value of the estimated credit with the actual credit and may request additional detail on the credit from the manufacturer to explain any discrepancies between the estimate and the actual credit. In another embodiment, the warranty tracking unit 114 may request additional credits based on the warranty information. In step 422, the credit calculation unit 116 determines the payees of the credit based on predefined rules associated with the warranty. In one embodiment, the predefined rules are generated by the client. In another embodiment, the predefined rules are provided as part of the warranty information. In another embodiment, the warranty information is provided by a third party such as an insurance company.

In step 424, the credit calculation unit 116 retrieves payment rules related to each payee. The payment rules may include the method of payment, the time period to receive payment or any other information related to the payment of the credit. In step 426, the payment distribution unit 118 determines the amount to pay each payee based on the payment rules. In step 428, the payment distribution unit 118 generates payments to the payees based on the payment rules. In one embodiment, the payment distribution unit 118 may determine the total amount of payments made to a payee over a predetermined time period.

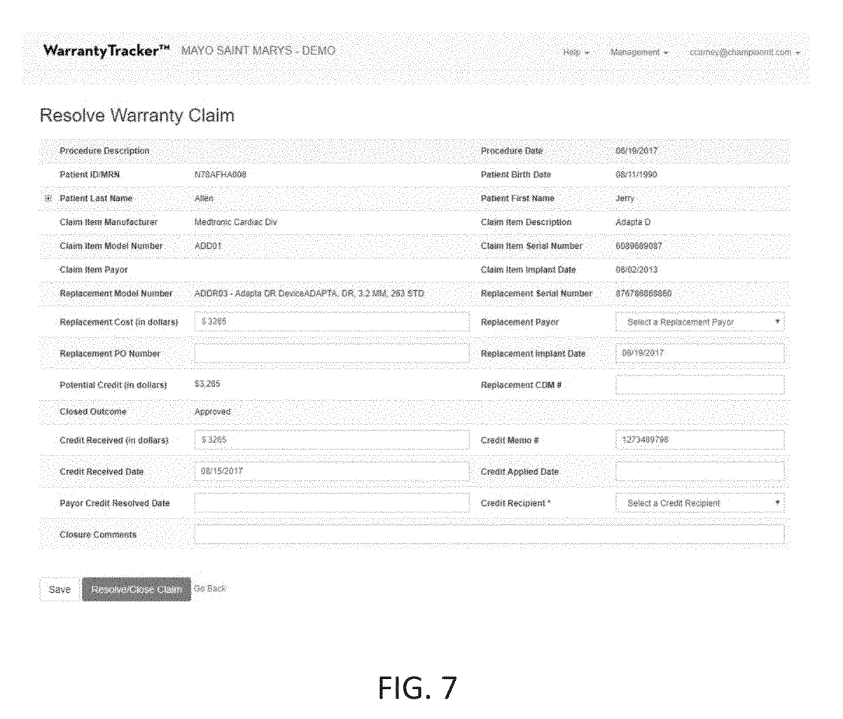

In an embodiment, the display device 206 may display the status of a claim to a user using the GUI on the display device 206 and enable the user to elect whether to close or resolve a claim manually. An exemplary depiction of the GUI displayed on the display device 206 is provided in FIG. 7. As shown, the GUI presents the user with information relating to the claim, including the outcome of the claim (approved or denied), the credit received (if any) and other information relating to the credit, such as the date on which it was received, a memorandum tracking number, and the recipient of the credit. Optionally, the user may input comments regarding the claim (such as notes to be used in connection with future claims).

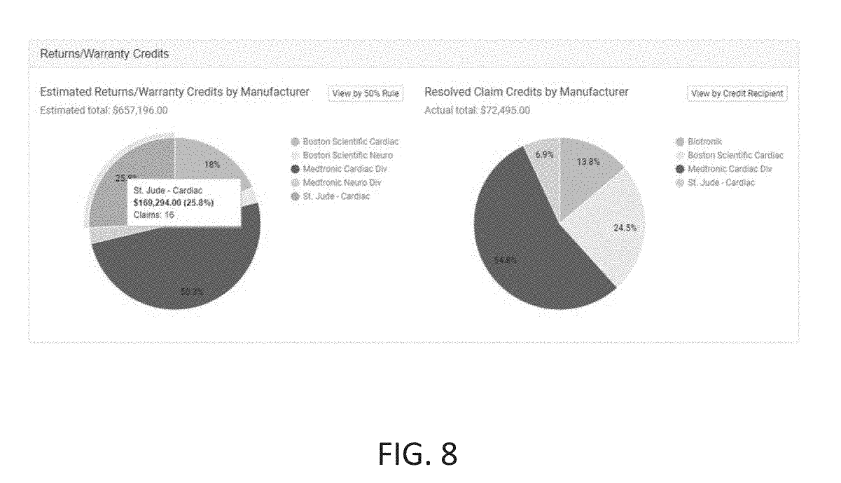

The warranty tracking unit 114 may also perform analytics regarding claims, including comparing the expected credits for claims to the actual amounts received from the manufacturer. This information may then be presented to a user via the GUI on the display device 206, as shown for example in FIG. 8. As will be clear to one of skill in the art, other analytical functions may also be performed by the warranty tracking unit 114 so as to enable users to understand the status of all processed and outstanding claims.

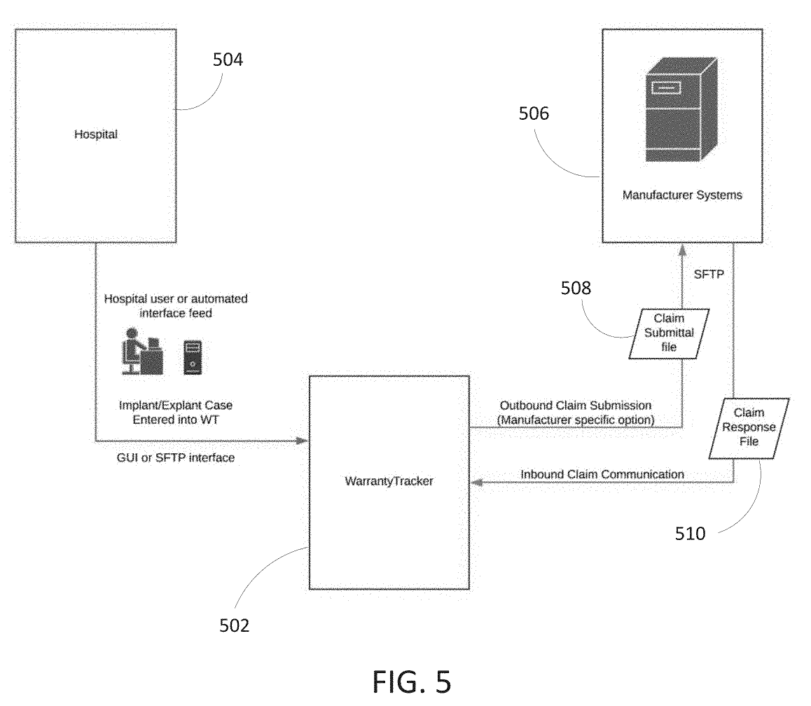

FIG. 5 depicts an exemplary schematic diagram of the communications between an exemplary warranty tracking unit 502, a hospital 504, and a manufacturer 506. As shown, the warranty tracking unit 502 is in a geographically distinct location from both the hospital 504 and the manufacturer 506. A user at the hospital 504 may access the warranty tracking unit 502 using a computer 104, which displays a GUI based on information received from the warranty tracking unit 502. In an alternative embodiment, a system at the hospital 504 such as computer 102 automatically accesses the warranty tracking unit 502, for example by using a secure file transfer protocol (SFTP) interface. Information regarding a medical device implanted with a patient is sent from the hospital 504 to the warranty tracking unit 502.

The warranty tracking unit 502 also communicates with a manufacturer 506. The warranty tracking unit 502 transmits a claim submittal file 508 to the manufacturer 506 in the format required by the manufacturer 506. In an embodiment, the requisite format for the claim submittal file 508 is automatically determined by querying the manufacturer 506. In an embodiment, the claim submittal file 508 contains information regarding the medical device (e.g., manufacturer name, model number, description, and serial number), information regarding the date on which the medical device was implanted, the status of the medical device (i.e., whether it was removed or replaced), and the date on which the medical device was removed from service.

After receiving the claim submittal file 508, the manufacturer 506 transmits a claim response file 510 to the warranty tracking unit 502 once the submitted claim has been processed. The claim response file 510 contains information regarding the outcome of the claim (i.e., accepted or rejected) and the amount of any credit that will be paid by the manufacturer 506.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed is:

1. A warranty management system, the warranty management system comprising a processor communicatively coupled to a non-transitory memory, a network link, and a wireless transmitter, the non-transitory memory containing a program configured to call the processor to process a warranty claim by executing the steps of:
gathering medical device information from a medical device communicatively coupled to the warranty management system via the wireless transmitter, wherein the medical device information includes a serial number for a medical device associated with the warranty claim;
retrieving warranty information from the medical device, wherein the warranty information to be retrieved is determined based on the medical device information;
determining, based on the warranty information, whether the medical device is covered by a warranty and calculating a credit amount due based on the warranty;
generating a claim submittal file for the warranty claim based on the warranty information and the credit amount;
transmitting the claim submittal file to a manufacturer of the medical device via the network link;
receiving a confirmation file from the manufacturer via the network link that the claim submittal file was successfully received by the manufacturer, wherein the confirmation file includes an identifier for the warranty claim;
storing the identifier in the non-transitory memory, wherein the identifier is associated in the non-transitory memory with the claim submittal file;
receiving a claim response file for the warranty claim from the manufacturer via the network link, wherein the claim response file is associated with the claim submittal file based on the identifier and the claim response file includes at least a payment amount for the warranty claim;
retrieving a plurality of payment rules for the warranty claim from the non-transitory memory, wherein the plurality of payment rules comprises at least one payee and a predetermined payment period; and
distributing the payment amount to the at least one payee over the predetermined payment period based on the plurality of payment rules.

2. The warranty management system of claim 1, wherein the program is further configured to call the processor to process the warranty claim by causing the processor to execute the steps of:
after receiving the confirmation file, monitoring the status of the warranty claim and transmitting a plurality of status requests to the manufacturer via the network link, wherein each of the plurality of status requests is transmitted at a predetermined, regular interval; and
after receiving the claim response file, notifying a user of the payment amount via the network link.

3. The warranty management system of claim 1, wherein the plurality of payment rules further comprises a first payee, a second payee, and a percentage owed to each of the first payee and the second payee, wherein the program is further configured to call the processor to process the warranty claim by causing the processor to execute the step of distributing the percentage owed to the first payee of the payment amount to the first payee and the percentage owed to the second payee of the payment amount to the second payee.

4. The warranty management system of claim 1, wherein the program is further configured to call the processor to process the warranty claim by causing the processor to execute the step of gathering:
displaying the credit amount to a user via the display;
obtaining confirmation from the user via the input device that the credit amount is correct;
displaying the warranty information to the user via the display;
obtaining confirmation from the user via the input device that the claim submission file may be submitted to the manufacturer; and
notifying the user via the display that a claim response file has been received by the manufacturer.

5. The warranty management system of claim 4, wherein the warranty management system further comprises a display and an input device and the program is further configured to call the processor to process the warranty claim by causing the processor to execute the steps of:
gathering analytics information regarding a plurality of previously processed warranty claims from the non-transitory memory, wherein each of the plurality of previously processed warranty claims was previously processed by the warranty management system and the analytics information includes, for each of the previously processed warranty claims a respective credit amount, at least a paid amount, a manufacturer name, and a medical device identifier;
analyzing the analytics information to determine one or more payment trends;
generating a visualization of the one or more payment trends; and
presenting the visualization to the user via the display.

6. A warranty management system including:
an information gathering unit communicatively coupled to a medical device that is configured to gatherer identifying information from the medical device;
a warranty tracking unit communicatively coupled to a database containing a plurality of warranty information, a manufacturer, and the information gathering unit, wherein the warranty tracking unit is configured to gather warranty information for the medical device from the database based on the identifying information, to generate a warranty claim submission file based on the warranty information for the medical device, to transmit the warranty claim submission file to the manufacturer, and to receive a warranty claim response file from the manufacturer;
a credit calculation unit communicatively coupled to the warranty tracking unit configured to receive the warranty information for the medical device, to calculate the amount of a credit owed by the manufacturer based on the warranty information, and to provide the amount of the credit owed to the warranty tracking unit for inclusion in the warranty claim submission file; and
a payment determination unit communicatively coupled to the warranty tracking unit and configured to generate a list of payees based on the warranty information for the medical device and a payment amount for each payee.
